(19) United States
(12) Patent Application Publication
(10) Pub. No.: US 2010/0010563 A1
(43) Pub. Date: Jan. 14, 2010

(54) Method and Apparatus for Treating Fecal Incontinence

(75) Inventor: John Furness, Victoria (AU)

Correspondence Address: JONES DAY, 222 EAST 41ST ST, NEW YORK, NY, 10017, US

(73) Assignee: Continence Control Systems International Pty Ltd., Chatswood, New South Wales (AU)

(21) Appl. No.: 12/083,518

(22) PCT Filed: Oct. 13, 2006
(86) PCT No.: PCT/AU2006/001504

§ 371 Date: Aug. 26, 2009

(30) Foreign Application Priority Data
Oct. 14, 2005 (AU) 2005905673

(51) Int. Cl. A61N 1/36; A61F 2/04; A61F 2/48

(52) U.S. Cl. 607/41; 600/30

(57) ABSTRACT

The present invention relates to a method and apparatus for treating fecal incontinence. A smooth muscle sphincter is implanted about the distal part of the large intestine in the region of internal anal sphincter or the anal canal, the rectum or the sigmoid colon. The smooth muscle sphincter is electrically stimulated by a stimulator device, in order to contract the smooth muscle sphincter and maintain continence. Stimulation is ceased or varied in order to allow the smooth muscle sphincter to relax and the patient to defecate.

U.S. Pat. No. 6,659,936 issued on 9 Dec. 2003, International Patent Application PCT/AU00/00925 filed on 4 Aug. 2000, Australian Provisional Application AU PQ2026, filed on 4 Aug. 1999, relate to the control of continence. International Patent Application PCT/AU2005/001698 filed on 8 Nov. 2005, Australian Provisional Application AU2004906393, filed on 8 Nov. 2004, relate to an implantable electrode arrangement.

International Patent Application PCT/AU2006/001301 filed on 4 Sep. 2006, Australian Professional Application AU2005904830, filed on 2 Sep. 2005, relate to an implant for managing a medical condition.

Australian Provisional Application No: 2005905673 relates to a method and apparatus for treating fecal incontinence.

Each one of the above documents are incorporated herein by reference in their entirety.

### FIELD OF THE INVENTION

The present invention relates to a method and apparatus for treating fecal incontinence.

### BACKGROUND OF THE INVENTION

Fecal incontinence is a major medical problem which is an extremely debilitating condition for an affected individual. Otherwise healthy individuals may be effectively prevented from engaging in normal society. It has been estimated that up to 50% of occupants of Nursing Homes in the USA have been placed there, in part, as a result of a fecal incontinence condition. If the condition could be adequately addressed there is the potential to bring major advantages to the individual and society.

There are a number of causes of fecal incontinence, but a major cause is probably failure of nerve control of the internal and/or external fecal sphincters. Injury to the spinal cord, such as found in quadriplegics and paraplegics, often leads to fecal incontinence. Other causes may include failure of the sphincteric muscles. Causes may be age related, and there is a higher incidence of the condition in an ageing population.

A number of treatments have been proposed, including the use of artificial sphincters (inflatable sphincters), tissue bulking, graciloplasty and neuromodulation (low level stimulation of nervous pathways to modify the response of reflex pathways). None of these treatments has been found to be wholly effective.

In an earlier patent application, International Patent Application No. PCT/AU00/00925 (referred to above), a method and apparatus is proposed for treating urinary incontinence which includes the steps of forming a “neosphincter” from smooth muscle tissue taken from elsewhere in the patient's body, and wrapping the neosphincter around the urethra. An implantable stimulator provides an electrical signal to the neosphincter via an electrode or electrodes. The electrical signal stimulates the neosphincter to maintain tone about the urethra to reduce leaks from the bladder until the user wishes to urinate. A signal from a control device may cause the stimulator to stop providing the electrical signal to the neosphincter, to allow the neosphincter to relax and enable the individual to urinate. The stimulation may activate the muscle directly, or activate it through the excitation of nerve fibres that innervate the muscle.

### SUMMARY OF THE INVENTION

In accordance with a first aspect, the present invention provides an apparatus for treating fecal incontinence, the apparatus comprising a stimulator arranged to provide a signal for stimulation of contractile tissue, in order to facilitate fecal continence.

In an embodiment, the stimulator is arranged to be implanted within a patient. In an embodiment, the entire stimulator may be implanted within a patient. In another embodiment, a part of the stimulator may be implanted in the patient, and a part external.

In an embodiment, the stimulator may be external to the patient and provide stimulation signals across the skin to stimulate the contractile tissue.

In an embodiment, the contractile tissue is positioned proximate to the colorectum or fecal canal.

In an embodiment, the contractile tissue may be formed as a sphincter wrapped around the outside of the fecal canal or rectum, either proximate to the perineum or within the pelvis or abdomen.

In an embodiment, the contractile tissue is not skeletal muscle tissue.

In an embodiment, the contractile tissue may have properties the same as or similar to smooth muscle tissue.

In an embodiment, the contractile tissue may be formed from the internal fecal sphincter, and stimulation is applied to the internal fecal sphincter.

In an embodiment, the sphincter may be formed from the dartos muscle.

In an embodiment, the sphincter may be formed from muscle from the wall of the gastro intestinal tract.

In an embodiment, the contractile tissue may be smooth muscle tissue. The smooth muscle tissue may be transplanted tissue taken from a donor, from elsewhere in the patient's body, or may have been grown externally.

In an embodiment, the signal may be in the form of a pulsed signal, arranged to maintain tone in the contractile tissue to maintain closure of the lumen of the distal part of the large intestine.

In an embodiment, the distal part of the large intestine is the region of the internal fecal sphincter.

In an embodiment, the distal part of the large intestine is the fecal canal, the rectum or the sigmoid colon.

In an embodiment, in order to enable a patient to defecate, the stimulator may be arranged to provide a different stimulation signal or no stimulation signal in order to cause or allow the contractile tissue to relax and open the fecal canal. A controller, operable by the patient, may be provided to vary the stimulation signal to enable defecation. The advantage of such an arrangement is that a patient suffering from fecal incontinence may be able to maintain continence and also control the time of defecation.

Where the contractile tissue is smooth muscle, an advantage is that innervated smooth muscle requires only low amounts of power in order to maintain contractile tone. Also, as compared with skeletal muscle, it does not tire as easily and is able to maintain contraction for a longer period of time.

In an embodiment, the signal is arranged to indirectly stimulate the contractile tissue through stimulation of nerve fibres innervating the contractile tissue.

In accordance with a second aspect, the present invention provides a device for internal fecal sphincter, rectum or colon, and arranged to be stimulated to contract to facilitate fecal continence.

In an embodiment, the contractile tissue is positioned proximate to the colorectum or fecal canal.

In an embodiment, the contractile tissue is formed into a sphincter.

In an embodiment, the sphincter is positioned about the rectum.

In an embodiment, the sphincter is positioned about the fecal sphincter.

The contractile tissue in one embodiment is smooth muscle tissue.

In accordance with a third aspect, the present invention provides a controller for controlling a stimulator which is arranged to stimulate contractile tissue to facilitate fecal continence, the controller being arranged to provide a signal to the stimulator to vary the stimulation provided by the stimulator.

In an embodiment, the controller is arranged to provide a signal which causes the stimulator to provide no stimulation signal to the contractile tissue, resulting in the contractile tissue relaxing to allow a patient to defecate.

In an embodiment, the controller is arranged to generate a wireless signal to be received by a receiver associated with the implantable stimulator.

In accordance with a fourth aspect, the present invention provides a programmer for programming operation of a stimulator which is arranged to stimulate contractile tissue to facilitate fecal continence, the programmer including an interface enabling communication with the stimulator for programming of the stimulator.

In an embodiment, the programmer may be utilised by a clinician to set stimulation signal parameters of the stimulator.

In accordance with a fifth aspect, the present invention provides a system for treating fecal incontinence, the system comprising an apparatus in accordance with the first aspect of the invention and a device in accordance with the second aspect of the invention.

In an embodiment, the system further comprises a controller in accordance with the third aspect of the invention.

In an embodiment, the system further comprises a programmer in accordance with the fourth aspect of the invention.

In accordance with a sixth aspect, the present invention provides a system for treating fecal incontinence, comprising an apparatus in accordance with the first aspect of the invention and a controller in accordance with the third aspect of the invention.

In an embodiment, the system further comprises a programmer in accordance with the fourth aspect of the invention.

In accordance with the seventh aspect the present invention provides a system for treating fecal incontinence, comprising an apparatus in accordance with the first aspect of the invention and a programmer in accordance with the fourth aspect of the invention.

In accordance with an eighth aspect, the present invention provides a system for treating fecal incontinence, comprising a controller in accordance with the third aspect of the invention and a programmer in accordance with the fourth aspect of the invention.

In accordance with a ninth aspect, the present invention provides a method of treating fecal incontinence, comprising the steps of stimulating contractile tissue positioned about the colorectum or fecal sphincter of a patient in order to cause the contractile tissue to contract, by way of providing a stimulation signal to an electrode arranged to transmit the signal to the contractile tissue.

In an embodiment, the method comprises the further step of providing a further signal, or absence of a signal, in order to enable or cause the contractile tissue to relax and allow the patient to defecate.

In accordance with a tenth aspect, the present invention provides a method of treating fecal incontinence in a patient, comprising the step of implanting into the patient a stimulator device arranged to provide stimulation signals to contractile tissue in order to cause the tissue to contract to facilitate closure of the colon.

In an embodiment, the method comprises the further step of implanting the contractile tissue. The contractile tissue in one embodiment is in the for n of smooth muscle tissue.

In an embodiment, the contractile tissue is formed into a sphincter about the colon.

In an embodiment, the method includes the step of implanting a contractile tissue sphincter in the perineal position about the fecal canal.

In an alternative embodiment, the method includes the steps of implanting a contractile tissue sphincter about the rectum or colon in the abdomino-pelvic region.

In accordance with an eleventh aspect, the present invention comprises a method of treating fecal incontinence, comprising the steps of implanting contractile tissue in a position proximate to the colorectum, the contractile tissue being arranged to be stimulated to facilitate closure of the colorectum to maintain continence.

In an embodiment, the contractile tissue is formed into a sphincter about the fecal canal or fecal sphincter.

In an embodiment, the contractile tissue is smooth muscle tissue.

### BRIEF DESCRIPTION OF THE DRAWINGS

Features and advantages of the present invention will become apparent from the following description of embodiments therefore, by way of example only, with reference to the accompanying drawings, in which:

FIG. 1 is a schematic sagittal cross-section through the pelvic region of a patient illustrating an implanted system in accordance with one embodiment of the present invention;

FIG. 2 is a schematic sagittal cross-section through the pelvic region of a patient, illustrating an implanted system in accordance with a further embodiment of the present invention;

FIG. 3 is a block diagram of componentry of an implantable stimulator of the systems of FIG. 1 and FIG. 2;

FIG. 4 is a block diagram of a system in accordance with an embodiment of the present invention;

FIG. 5 is a block diagram of a further system in accordance with an embodiment of the present invention;

FIG. 6 is a schematic cross-section through the pelvic region of a patient illustrating an implanted system in accordance with a further embodiment of the present invention;

FIGS. 7, 8 & 9 are exploded perspective, plan and side views, respectively, of an electrode arrangement for delivering stimulation signals in a system in accordance with an embodiment of the present invention;

FIGS. 10, 11, 12 & 13 are perspective, plan, side section, detail views of a shroud component of the electrode arrangement of FIG. 7;

FIGS. 15, 16, 17, 18, 19, are perspective, rear, plan section, side section and plan views of a cover component of the electrode arrangement of FIG. 7.

### DETAILED DESCRIPTION OF PREFERRED EMBODIMENTS

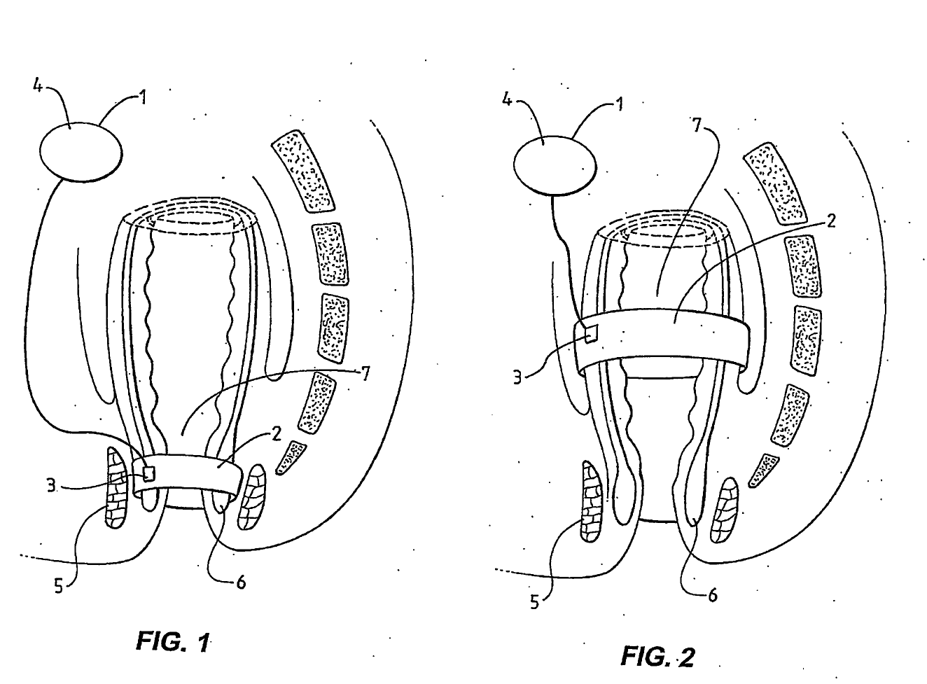

Referring to FIG. 1, a system and apparatus in accordance with an embodiment of the present invention, for treating fecal incontinence, are illustrated in schematic form. The system includes an apparatus comprising an implantable stimulator 1 and a device comprising contractile tissue 2 which is arranged to be stimulated by a signal that is generated by the stimulator 1 and, in this embodiment, applied to the contractile tissue 2 via an electrode 3 conductively connected between the stimulator 1 and contractile tissue 2.

In this embodiment, the stimulator 1 includes a signal generator for producing a pulsatile signal which is housed in a bio-compatible housing 4. The stimulator 1 will be described in more detail later.

The contractile tissue 2 in this embodiment is formed into a sphincter which is implanted about the fecal sphincter region, in this embodiment proximate to the anus. In FIG. 1, the external fecal sphincter is designated by reference numeral 5 and the internal fecal sphincter by reference numeral 6. Failure of operation of the external and/or internal fecal sphincters perhaps because of nerve damage, or other reason) have led to fecal incontinence in this patient. Stimulation of the contractile tissue sphincter 2, in operation, causes the contractile tissue 2 to contract and maintain closure of the fecal canal 7, maintaining fecal continence.

In this embodiment, the contractile tissue is smooth muscle tissue. The smooth muscle tissue may be obtained from elsewhere in the body, formed into a sphincter and surgically implanted. Alternatively, the smooth muscle tissue may be grown from smooth muscle stem cells and/or proliferative smooth muscle cells. Alternatively, the smooth muscle tissue may be transplanted smooth muscle tissue augmented by smooth muscle stem cells and/or proliferative smooth muscle cells. Alternatively, the smooth muscle tissue may be the tissue of the internal fecal sphincter.

International Patent Application No: PCT/2006/001301, referred to above, discloses augmentation of contractile tissue using proliferative smooth muscle cells or smooth muscle stem cells. Growth, maturation and stability of the tissue may be influenced by growth factors (trophic and/or neurotrophic factors) that are a component of the treatment.

Smooth muscle may be taken from anywhere or grown (as discussed above). In an embodiment the smooth muscle may be taken from the smooth muscle of the bladder and transplanted about the urethra, with its circulation intact. Alternatively, the muscle is venous smooth muscle, anococcygeus smooth muscle or terminal ileum transplanted as a segment devoid of mucosa and having its circulation intact. A further alternative is the dartos smooth muscle from the scrotum or a portion of the vagina or labia.

In an embodiment, smooth muscle may be taken as a free graft. In this case, the tissue is separated from its normal circulation and becomes vascularised by ingrowth of blood vessels at the site of implant.

The stimulator 1 includes a signal generator arranged to provide a stimulation signal for stimulating the smooth muscle sphincter 2. A lead 8 extends from the stimulator 1 to the electrode 3 at the smooth muscle sphincter 2, for providing the stimulation signal 2 to the smooth muscle sphincter 2. The stimulation signal may be a signal of frequency and amplitude determined to maintain contraction of the smooth muscle sphincter 2 to facilitate continence.

The stimulator 1 may also be arranged to produce a further electrical signal to stimulate the sphincter 2 to relax, to enable the patient to defecate. As an alternative to a further electrical signal, the stimulator 1 may be arranged to stop producing any electrical signal and it is the absence of the signal that causes the sphincter 2 to relax. In this embodiment, the stimulator 1 is arranged to have the stimulation signal varied under control of the patient by way of an external controller.

FIG. 2 shows an alternative embodiment. In the FIG. 2 drawing, the same reference numerals have been used as in FIG. 1 for equivalent components. Those components have the same function as in FIG. 1 and no further description will be given here. In the FIG. 2 embodiment, the contractile tissue sphincter 2 is placed further up the colorectum, in the abdomino-pelvic region, away from the anus.

This different positioning may be used if surgically convenient. In some cases, this different position may be utilised where there is some damage to the anus. Such damage may occur, for example, from the former use of prosthesis in an attempt to correct the incontinence problem. There does not have to be any damage to the anus for this alternative positioning to be used.

In a further alternative embodiment, the sphincter 2 may be positioned about the external fecal sphincter.

In a further alternative embodiment, a neosphincter may not be utilised, instead stimulation may be applied directly to the internal fecal sphincter 6.

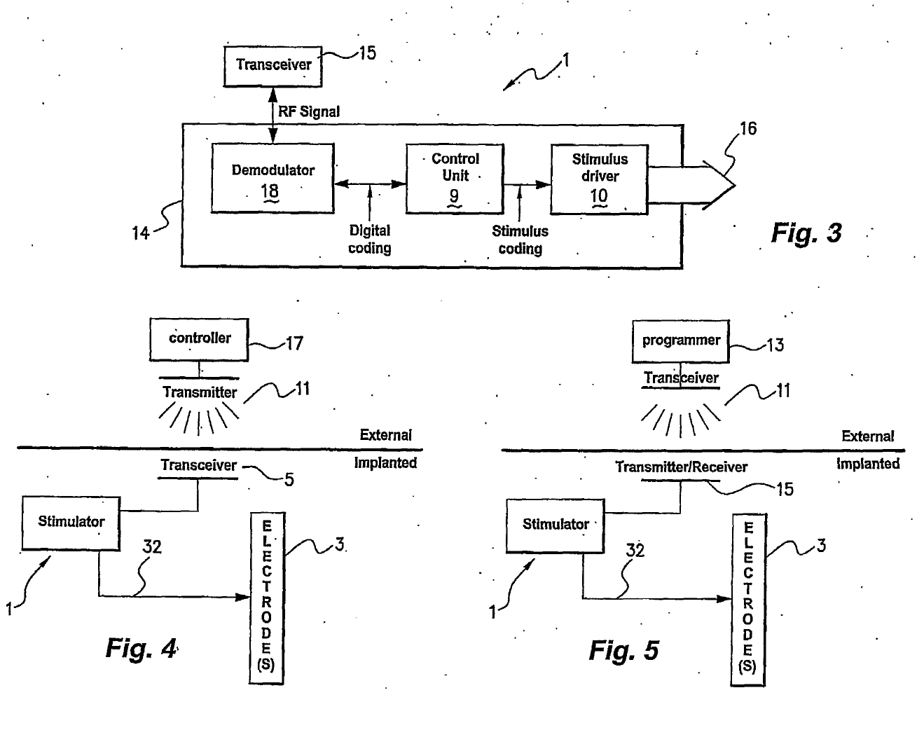

The stimulator 1 is shown in more detail in FIG. 3. In this embodiment, a signal generator that is arranged to provide the electrical signal for stimulation of the sphincter 2 is in the form of a control unit 9 and stimulus driver 10. The control unit 9 encodes the stimulus and provides a signal to the stimulus driver 10 which provides the stimulation signal at output 16. The output 16 outputs to conductor 32 and to one or more electrodes 3.

In this embodiment, the control unit 9 and stimulus driver 10 form, together with a demodulator 18, a processing unit for generating the stimulation signal(s) at output 16.

The demodulator 18 is arranged to demodulate a signal received by transceiver 15. An external control unit and external programmer unit (both to be described later) are able to communicate via the transceiver 15 with the processing unit 14 in order to control application of stimuli and/or vary the stimuli. In addition, as described in more detail later, the processing unit 14 may transmit, via control unit 9, demodulator 18 and transceiver 15, signals to the control unit or programmer unit. The transmitted signals may deliver telemetry information indicative of parameters of the stimulator, for the purposes of calibration and control.

The entire stimulator 1 (including components 14 and 15), is enclosed in a housing which includes a casing made from a bio-compatible material, such as titanium, silicone polymer or other acceptable materials, or combinations of materials, including, but not limited to inert materials. The frequency of the RF signal for transmission and reception by the transceiver 15 may depend on the material of the casing of the stimulator.

FIG. 4 shows a system in accordance with an embodiment of the present invention. The system incorporates the implanted stimulator 1, with transceiver 15. The electrode(s) 3 is shown schematically together with cable 32.

The system also comprises an external controller 17 which includes a transmitter 11. The controller 17 is intended for operation by a patient with the stimulator implanted, for control of the stimulator 1.

The controller 17 includes an actuator (such as a button, not shown) operable by the patient to selectively send signals to the implanted stimulator 1, for control of the stimulation signals being sent to the electrode(s) 3.

In one embodiment, the stimulator is “fail safe”. Unless a signal is received from the controller 17, the stimulator produces a signal which maintains tone in the smooth muscle implant 2, maintaining fecal continence.

When the patient wishes to defecate, the patient actuates the controller 17 to send, via the transmitter 11, a signal to the stimulator 1. In response to receiving the signal, the control unit 9 operates to turn the stimulating signal off causing the sphincter 2 to relax and allow the patient to defecate.

The controller 17 may also be arranged to provide a further signal under patient control, once the patient has finished defecating, the further signal causing stimulator 1 to resume providing the stimulation signals to the electrode(s) 3.

In “fail safe” mode, if the further signal is not produced, the stimulator may resume providing the stimulation signal to the electrode(s) 3 after a predetermined period of time.

The stimulation signal 16 provided to contract the smooth muscle sphincter 2 is selected so as to provide a substantially continuous tone in the sphincter 1. A generally rectangular and symmetrically biphasic pulse may be suitable for this. The signal has a substantially constant current less than or equal to 50 mA, 15 mA, 10 mA, or 5 mA, and in some preferred embodiments may be in the order of 4 mA, 8 mA, 12 mA, or 15 mA.

Stimulation pulse frequency provided to sphincter 1 is in the range of 0.1 Hz to 5 Hz, 0.2 Hz to 4.0 Hz. 0.25 Hz to 3.0 Hz, 1 Hz to 3.0 Hz, 1.5 Hz to 3 Hz, 1.75 Hz to 2.5 Hz, or a 0.25 Hz to 2.25 Hz, and in one embodiment, is 1 Hz, 2 Hz, 2.5 Hz or 3 Hz. Stimulation phase width of each phase is in the range of 0.05 ms to 2.0 ms, 0.1 ms to 1.5 ms, 0.2 ms to 1 ms, 0.25 ms to 0.75 ms, and in one embodiment is 0.2 ms, 0.4 ms, 0.5 ms or 1 ms. The stimulator is current regulated, and accordingly the stimulation voltage will vary with the resistance of the muscle tissue between the electrodes.

Typical values for the voltage are between 0.1 and 15 Volts, 0.2 and 12 Volts, 0.5 and 12 Volts, 0.5 and 10 Volts, or 0.5 and 7.5 Volts. In one embodiment, the voltage is 2.5 Volts, 5 Volts, 7.5 Volts or 10 Volts. Either a current source (voltage limited) or a voltage source (current limited) stimulator may be used.

It is also possible to use an asymmetric biphasic pulse, in which, for example, the first phase is shorter in duration than the second phase.

FIG. 5 shows a system in accordance with an embodiment of the present invention, including a programmer unit 13 which may be utilised by a physician to set and adjust parameters of the implanted stimulator 1. The programmer unit is arranged for communication with the stimulator via transceiver 11, and may comprise a computing device. The control unit 9 is also arranged to transmit stimulator telemetry information indicative of one or more of the parameters of the stimulator 1, for detection by the programmer 13 via transceiver 1. The programmer unit 13 can therefore determine parameters of the stimulator from telemetry information and can adjust the parameters by transmitting control signals to the stimulator 1. The signal from the programmer may be able to selectively vary the output current, shape, frequency and/or pulse width of the stimulation signal(s).

In operation, a physician adjusts parameters of the stimulation signal(s). The physician will note feedback from the patient as to the effect of the stimulus on fecal continence control, and may subsequently re-adjust the parameters until the stimulation is optimum. For example, patient perceived feedback may be used to set the maximum stimulation threshold of the smooth muscle sphincter.

In the above-described embodiments, signals between the controller or programmer and the stimulator are RF signals. Other types of transmission media other than RF may be used. For example, microwave signals may be used for transmission, optical signals may be used, and in another embodiment magnetic transmission may be used.

Magnetic transmission may be used for the controller 17 to cause the stimulator to stop producing stimulation signals and therefore allow the patient to defecate. In this embodiment, the controller 17 may be a simple magnet which, when passed over a magnetic receiver of the stimulator 1, results in the stimulator ceasing to provide stimulation signals for contracting the sphincter.

Other means than magnetic transmission may be utilised.

In the above embodiments, any suitable electrode(s) may be utilised to stimulate the implant 2. For example, button electrodes, cuff electrodes or any other suitable electrode may be utilised.

In embodiments, an electrode arrangement such as a disclosed in PCT/AU/20054/001698 may be utilised.

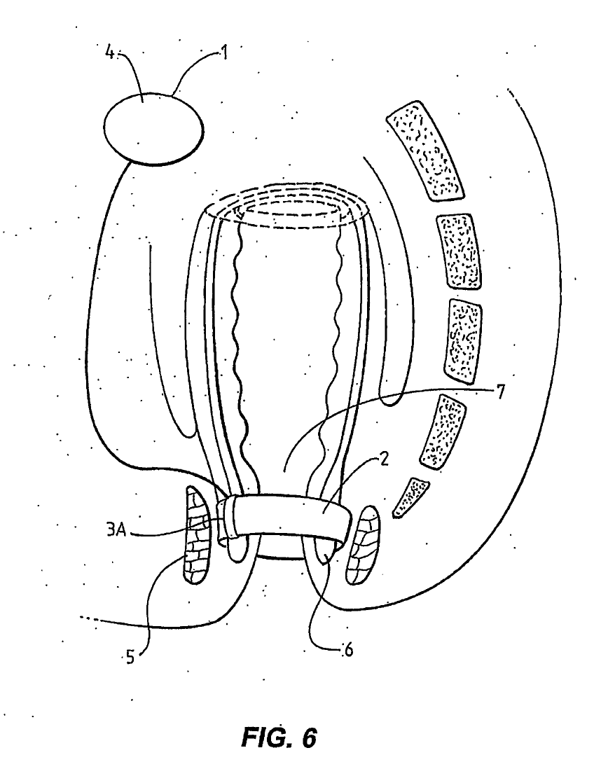

FIG. 6 illustrates an embodiment of the present invention where a “peg” electrode 3A such as disclosed in PCT/AU20054/001698 is utilised to transmit signals to the implant 2 from the stimulator 1.

In FIG. 6, the same reference numerals as used in previous embodiments have been utilised to designate similar components, and no further description will be given here of these components.

The electrode 3A will now be described in more detail.

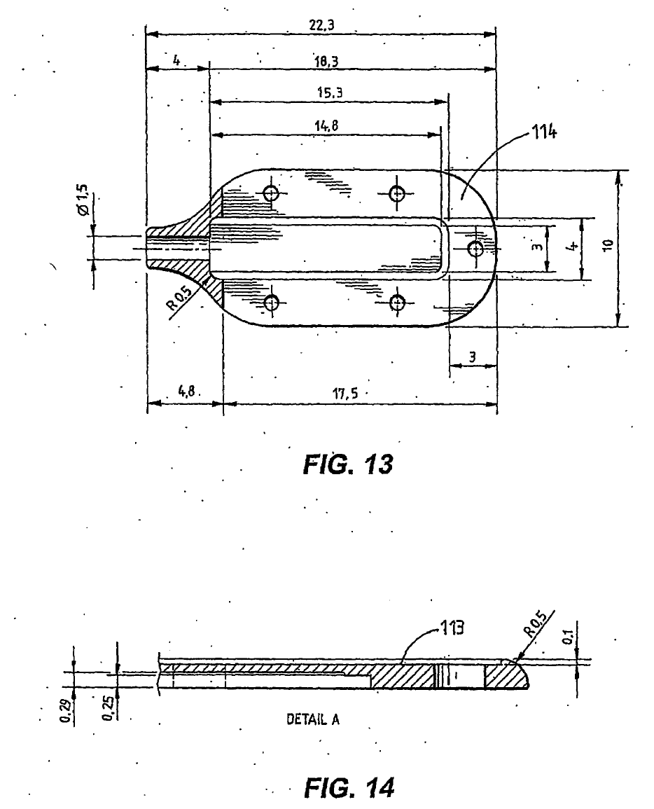

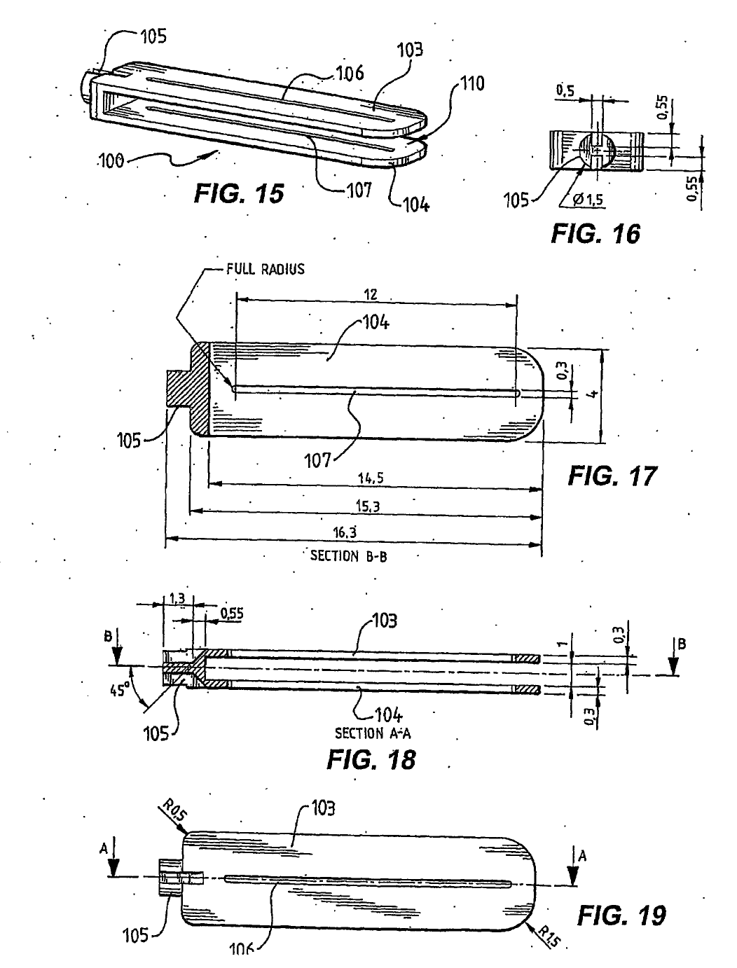

The electrode comprises a number of components. These include an electrode cover 100 (shown in most detail in FIGS. 14 through 18).

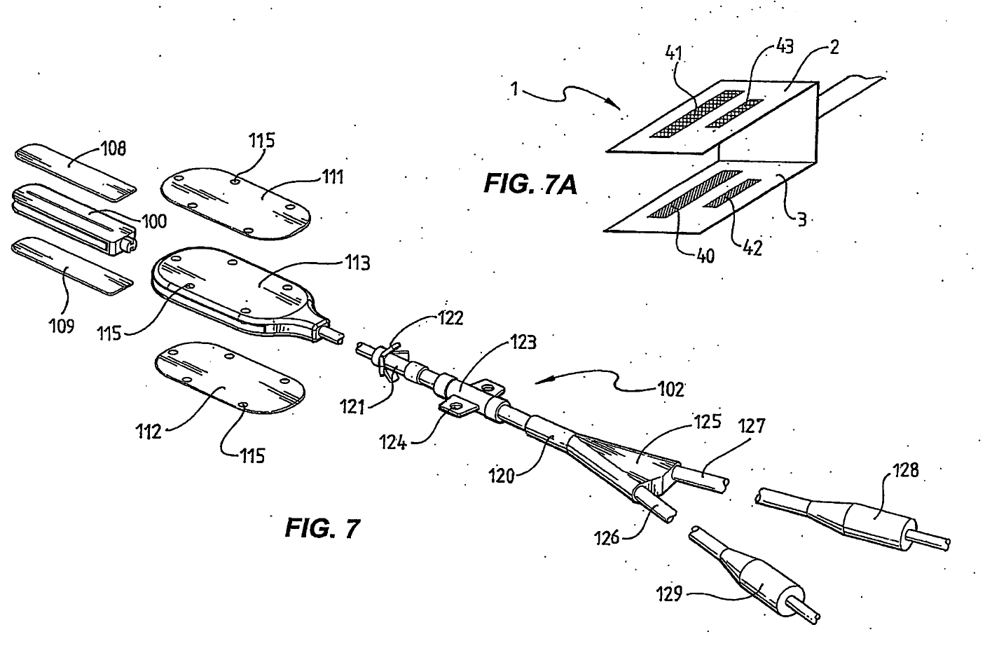

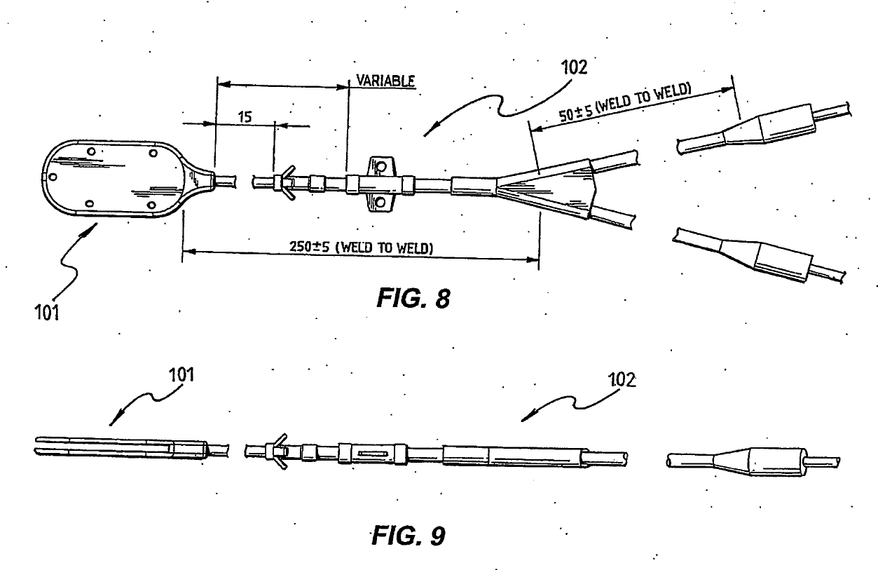

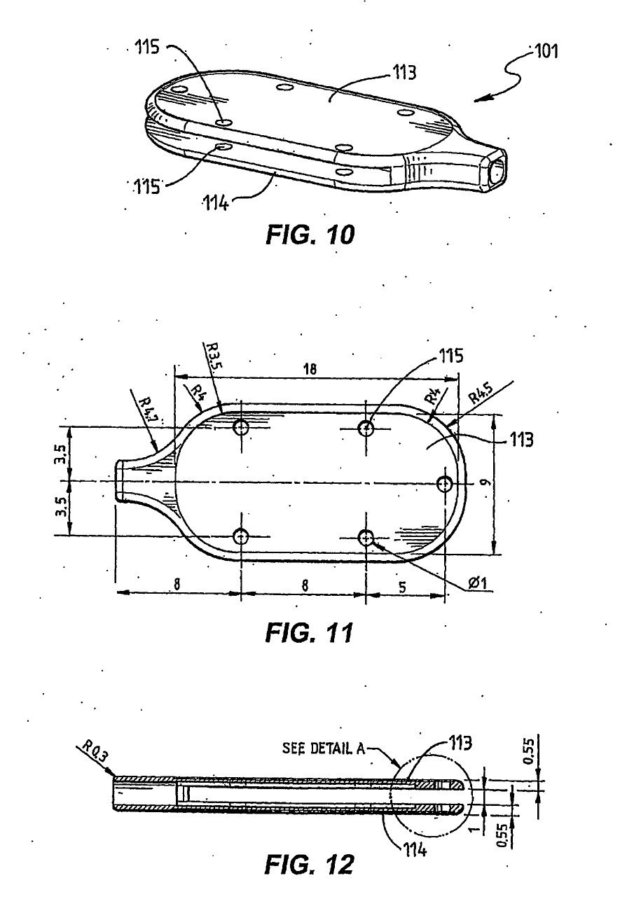

The components also include an electrode shroud (shown in best detail in FIGS. 10 through 13) and also an electrode lead 102 (shown in FIGS. 7, 8 & 9, together with the other components of the electrode arrangement).

In this embodiment first and second electrode elements are formed by the electrode cover 100, which includes insulating elements 103,104 extending from a base 105. The insulating extending elements 103,104 are formed with a slot 106,107, respectively, extending substantially along the length of the extending elements 103,104. When the electrode arrangement is assembled, platinum foil electrodes 108,109 (FIG. 7) are placed on the outer surfaces of the elements of the elements 103,104 so that they are insulated from the gap 110 formed between the elements 103,104 apart from the slots 106,107, which expose portions of the conductive plates 108,109 to the gap 110 (and, in use, to any tissue seated within the gap).

When assembled, the electrode cover 100 and platinum electrode foils 108,109 seat within the electrode shroud 101 as best shown in FIGS. 10, 11, 12 & 13. FIG. 13 in particular shown in cross-section where the electrode cover seats.

Electrode shroud 1 is formed from silicone. In order to provide reinforcement, PET mesh covers 111,112 are provided to fit to upper 113 and lower 114 extending portions of the shroud 101. Suture holes 115,116 are provided in the covers 111,112 and also in the elements 113,114 of the shroud 101. Note that the reinforcement can be provided by other means and is not limited to PET mesh. Further, the electrode shroud need not be in silicone but could be of other bio-compatible material and may not require reinforcement. Further, note that other means for affixing to the tissue may be provided other than suture holes or instead of suture holes.

The electrode lead 102 is a multi-component arrangement which includes an outer insulating cover 120, a tine collar 121 including tines 122 for retaining the lead in position within a patient. It also includes a sutured collar 123 including suture holes 124 for suturing to patient tissue to also facilitate retaining the lead 102 in position. There is also bifurcation moulding 125 which enables the lead to split into two parts 126,127 which may contain separate conductors, and connectors 128,129 which may be arranged to contact to a simulation device.

In the above embodiments, the electrode arrangement includes a pair of electrode elements which extend away from a base which joins them together at their proximal ends. In a further embodiment, a single electrode element which is not joined at any base is provided. This single electrode element may be used to provide stimulation to contractile tissue on its own, or may be used together with one or more similar electrode elements to provide stimulation.

In the above described embodiments, each electrode element is provided with a single electrode. The single electrode is an elongate electrode extending substantially the majority of the length of the electrode element.

One advantage of having thin electrodes bounded by insulating material on either side is that the arrangement operates to confine the electric field produced by the electrode to the tissue immediately adjacent the electrode. This reduces or prevents stimulation of tissue that it is not desirable to stimulate e.g. tissue external to a contractile tissue sphincter being controlled.

In operation, the electrodes 108, 109 and extending elements 103, 104 are positioned either side of the smooth muscle implant to enable signals to be transmitted to the implant for operation.

Electrode arrangement 3A allows application of an electric field between the opposing electrode elements to stimulate the tissue between them. The electric field in one embodiment is confined so that stimulation is to a band of tissue between the electrodes.

In one embodiment, innervation runs within the implant 2 perpendicular to the band of tissue being stimulated.

The elements in electrode 3A extend over the tissue in a manner analogous to that of a clothes peg.

The elements in electrode 3A extend over the tissue in a manner analogous to that of a clothes peg.

Other electrode patterns then a single line electrode on the surfaces of the elements may be utilised.

FIG. 7A discloses one alternate electrode pattern.

As discussed above, in an embodiment, the stimulater implant is preferably sealed and encased in a biologically inert material such as a biocompatible silicone material. Metallic electrodes and leads may be of plantinum-iridum alloy. The connecting wires are, in one embodiment, insulated with a silicon coating.

The implant may be placed between the abdominal muscle and the skin.

In the above embodiment, the stimulator is a totally implantable device. In an alternative embodiment, the stimulator may not be implantable. The stimulator in this embodiment may comprise a stimulator device having similar componentry to that discussed above in relation to the embodiment of FIGS. 3, 4 and 5, but being ranged to be placed externally of the patient. In one embodiment, signals are coupled to electrodes placed within the patient in order to stimulate the contractile tissue. Coupling may be by way of inductively coupling the signals across the patient's skin to an internally positioned electrode arrangement. In another embodiment, part of the stimulator componentry may be placed outside the patient and part inside the patient.

In the above embodiments a single stimulation signal generator is used to provide the electrical signal. Other embodiments may use two or more signal generators.

Other embodiments may use two or more stimulators, which may be placed in different locations.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to

1. An apparatus for treating fecal incontinence, the apparatus comprising a stimulator arranged to provide a signal for stimulation of implanted contractile tissues positioned proximate to one of the colon and rectum in order to facilitate fecal continence.

2. An apparatus in accordance with claim 1, wherein the contractile tissue is formed as a sphincter positioned about one of the colon, rectum and fecal sphincter.

3. An apparatus in accordance with claim 2, wherein the sphincter is positioned about the fecal sphincter.

4. An apparatus in accordance with claim 2, wherein the sphincter is positioned about one of the colon, rectum and fecal canal in the abdomino-pelvic region.

5. An apparatus in accordance with claim 1, wherein the contractile tissue is formed as a sphincter positioned about the external fecal sphincter.

6. An apparatus in accordance with claim 1, wherein the contractile tissue is smooth muscle tissue.

7. An apparatus in accordance with claim 1, wherein the signal is in the form of a pulse signal, arranged to maintain tone in the contractile tissue to maintain fecal continence.

8. An apparatus in accordance with claim 1, the implantable stimulator being arranged to provide a different stimulation signal or no stimulation signal in order to allow the contractile tissue to relax to enable the patient to defecate.

9. An apparatus in accordance with claim 1, the stimulator being an implantable stimulator and being arranged to be implanted within a patient.

10. A device for treating fecal incontinence, comprising implanted contractile tissue positioned proximate to one of the colon and rectum, and arranged to be stimulated to contract to facilitate fecal continence.

11. A device in accordance with claim 10, wherein the contractile tissue is in the form of a sphincter positioned about one of the colon and rectum.

12. A device in accordance with claim 11, wherein the sphincter is positioned about the fecal sphincter.

13. A device in accordance with claim 11, wherein the sphincter is positioned about the external fecal sphincter.

14. A device in accordance with claim 11, wherein the sphincter is positioned about one of the colon, rectum and fecal canal in the abdomino-pelvic region.

15. A device in accordance with claim 10, wherein the contractile tissue is smooth muscle tissue.

16. A controller for controlling a stimulator which is arranged to stimulate implanted contractile tissue positioned proximate one of the colon and rectum to facilitate fecal continence, the controller being arranged to provide a signal to the stimulator to vary the stimulation provided by the stimulator.

17. A controller in accordance with claim 16, wherein the controller is arranged to provide a signal which causes the stimulator to vary the stimulation to the contractile tissue, resulting in the contractile tissue relaxing to allow a patient to defecate.

18. A controller in accordance with claim 17, wherein the controller is arranged to provide a signal which causes the stimulator to provide no signal to the contractile tissue to enable it to relax.

19. A programmer for programming operation of a stimulator which is arranged to stimulate implanted contractile tissue positioned proximate one of the colon and rectum to facilitate fecal continence, the programmer including an interface enabling communication with the stimulator for programming of the stimulator.

20. A programmer in accordance with claim 19, the interface being arranged to enable setting of stimulation signal parameters of the stimulator.

21. (canceled)

22. (canceled)

23. (canceled)

24. (canceled)

25. (canceled)

26. (canceled)

27. (canceled)

28. A method of treating fecal incontinence, comprising the steps of stimulating implanted contractile tissue positioned proximate to one of the colon and rectum of a patient in order to cause the contractile tissue to contract, by way of providing a stimulation signal to an electrode arranged to transmit the signal to the contractile tissue.

29. A method in accordance with claim 28, comprising the further step of providing a further signal, or absence of a signal, in order to enable the contractile tissue to relax and enable the patient to defecate.

30. A method of treating fecal incontinence in a patient, comprising the step of implanting into the patient a stimulator device arranged to provide stimulation signals to implanted contractile tissue positioned proximate to one of the colon and rectum in order to cause the tissue to contract to facilitate closure of one of the colon, rectum and fecal sphincter region.

31. A method in accordance with claim 30, comprising the further step of implanting the contractile tissue.

32. A method in accordance with claim 31, wherein the step of implanting the contractile tissue comprises the step of implanting a contractile tissue sphincter in the perineal position about one of the colon and rectum.

33. A method in accordance with claim 31, wherein the step of implanting the contractile tissue comprises the step of implanting a contractile tissue sphincter about one of the colon and rectum in the abdomino-pelvic region.

34. A method of treating fecal incontinence, comprising the steps of implanting contractile tissue in a position proximate to one of the colon and rectum, the contractile tissue being arranged to be stimulated to facilitate closure of one of the colon, rectum and fecal sphincter region to maintain continence.

35. A method in accordance with claim 34, wherein the contractile tissue is formed as a sphincter about one of the colon, rectum and fecal sphincter region.

36. A method in accordance with claim 34, wherein the contractile tissue is smooth muscle tissue.

37. An apparatus for treating fecal incontinence, the apparatus comprising a stimulator arranged to provide a signal for stimulation of contractile tissue through stimulation of nerve fibres innervating the contractile tissue, in order to facilitate fecal continence.

38. (canceled)

39. (canceled)

40. (canceled)

41. A method of treating fecal incontinence, comprising the steps of stimulating innervated contractile tissue positioned proximate to one of the colon and rectum of a patient in order to cause the contractile tissue to contract, through stimulation of nerve fibres innervating the contractile tissue, by way of providing a stimulation signal to an electrode to arranged to transmit the signal to the contractile tissue.

42. (canceled)

43. (canceled)
